(19) United States
(12) Patent Application Publication
(10) Pub. No.: US 2015/0335911 A1
(43) Pub. Date: Nov. 26, 2015

(54) LIGHT EMITTING SYSTEM FOR WOUND CARE

(71) Applicants: Gary S. Rogers, Wenham, MA (US); Samuel L. Hill, Somerville, MA (US)

(72) Inventors: Gary S. Rogers, Wenham, MA (US); Samuel L. Hill, Somerville, MA (US)

(73) Assignee: Rogers Sciences, Inc., Beverly, MA (US)

(21) Appl. No.: 14/716,130

(22) Filed: May 19, 2015

Related U.S. Application Data
(60) Provisional application No. 62/000,770, filed on May 20, 2014.

(51) Int. Cl. A61N 5/06

(52) U.S. Cl. CPC A61N 5/0616; A61N 2005/0626; A61N 2005/063; A61N 2005/0652; A61N 2005/005

(57) ABSTRACT

A therapeutic light delivery system is provided that includes one or more fiber optic arrays that channels illumination from an illumination device. An optics module coupled to the one or more fiber optic arrays that provides the illumination to the one or more fiber optic arrays. A control module coupled to the optics module that controls the illumination to the one or more fibers. The illumination comprises a plurality of therapeutic illumination patterns and wavelengths.

### PRIORITY INFORMATION

This application claims priority from provisional application Ser. No. 62/000,770 filed May 20, 2014, which is incorporated herein by reference in its entirety.

### BACKGROUND OF THE INVENTION

The invention is related to the field of photodynamic therapy (PDT), and in particular to a light emitting system used in PDT.

In the medical device field there are numerous techniques to deliver light to perform a medical procedure, but the two most common techniques are direct and focused illumination. Direct illumination occurs with a bare or diffused light source placed a distance of several centimeters to meters from the patient. Direct Illumination devices are rarely attached to the patient. In general, the patient is required to position themselves to the illumination source. Examples of light delivery devices that fall within this category include conventional phototherapy units, such as the standard light box and hand/foot units that emit UV-A, UV-B or narrow-band UV-B light.

Phototherapy units are used primarily for the treatment of inflammatory skin diseases such as psoriasis. The units are also used in conjunction with orally or topically administered psoralens that photoactivate with UV-A light in the treatment of severe psoriasis and extensive vitilligo. This treatment is known as PUVA (psoralen UV-A) therapy. For systemic diseases such as cutaneous lymphoma, graft versus host disease and systemic sclerosis, extracorporeal photophoresis is performed where the patient ingests the psoralen and the blood is exposed to the UV-A light outside the body and then re-infused into the patient. The DUSA (blue visible light) and Galderma-Metvix (red visible light) systems are used for the treatment of actinic keratoses (pre-malignant skin growths) and superficial basal cell carcinomas. They work via topical aminolevulinic acid (DUSA) and methyl-aminolevulinic acid PDT.

Focused illumination, both internal and external to the patient treatment site requires illumination that has an optical system to direct light from the illumination device to specific areas onto the patient, typically in a controlled beam shape and beam intensity. In many cases the optical system is composed of one or more optical fibers that use total internal reflection to collect light at one end of the fiber, transmit the light, and exit with a specific numeric aperture at the other end. Typically this approach requires larger fibers or an array of large fibers to illuminate large areas (>5 mm). Illuminating more than a single fiber requires sophisticated coupling of the light into the fibers. This coupling is usually inefficient and can have very low coupling efficiency (<10% efficiency). Similar to direct illumination, the focused illumination approaches is rarely done where a patient wears a device.

For FDA approved PDT indications, there are numerous light illumination devices meeting the direct and focused illumination schemes. For example, for Barrett's esophageal cancer treated with PDT, a focused illumination system is implemented using a fiber optic cable attached to a FDA approved laser system such as the Angio Dynamics PDT 630 nm laser. Alternatively, a direct illumination approach to PDT for actinic keratosis is done using similar devices such as DUSA's Blue-Light Phototherapy Lamp or Galderma's Aktilite which is also used for basal cell carcinoma skin cancer.

There are few wearable medical based illumination devices except for the Ambicare Health Ambulight PDT device that only covers a small area and has no degree of flexibility or conformity to anatomical features. The device is a pad of LEDs that are placed directly on the treatment area. This method of delivery does allow the system to be portable, but it places the illumination source directly on the patient causing thermal side effects.

Another device that is wearable, but displaces the illumination source and any generated heat from the source at a distance from the treatment site is a weaved collection of fiber optic cables that are bent sharply at several locations along the length of the fiber. The bending of the fiber cause light to leak from the fiber illuminating a small portion of a light illumination surface that consists of hundreds to thousands of these bent fibers. This weaved fiber approach provides imprecise quantities of light at the treatment site because the bending (the mechanism of light leakage) of the fiber is not uniform from bend to bend and the location of bending along similarly aligned fibers can be random from fiber to fiber.

### SUMMARY OF THE INVENTION

According to one aspect of the invention, there is provided a therapeutic light delivery system. The therapeutic light delivery system includes one or more fiber optic arrays that channels illumination from an illumination device. An optics module coupled to the one or more fiber optic arrays that provides the illumination to the one or more fiber optic arrays. A control module coupled to the optics module that controls the illumination to the one or more fibers. The illumination comprises a plurality of therapeutic patterns and wavelengths.

According to another aspect of the invention, there is provided a method for delivering light for wound care. The method includes providing one or more fiber optic arrays that channels illumination from an illumination device. Also, the method includes coupling an optics module to the one or more fiber optic arrays that provides the illumination to the one or more fiber optic arrays. Furthermore, the method includes controlling the illumination to the one or more fibers using a control module coupled to the optics module. The illumination comprises a plurality of therapeutic patterns and wavelengths.

### BRIEF DESCRIPTION OF THE DRAWINGS

FIGS. 1A-1C are schematic diagrams illustrating the spring system, light emitting diode (LED) optics and the printed control board (PCB) used in accordance with the invention;

FIG. 2 is schematic diagram illustrating the die-layout options used in accordance with the invention;

FIG. 3 is a schematic diagram illustrating the canted-coil spring system used in accordance with the invention;

FIG. 4 is a schematic diagram illustrating an illumination unit (ID) used in accordance with the invention;

FIG. 5 is a schematic diagram illustrating the light emitting patch (LEP);

FIG. 6 is a schematic diagram illustrating a second embodiment of the LEP of FIG. 5;

FIG. 7 is a schematic diagram illustrating the LEP having a single fiber layer;

FIG. 8 is a schematic diagram illustrating the top view of the LEP having a single fiber layer;

FIG. 9 is a schematic diagram illustrating a LEP having a single fiber layer with bent fibers;

FIG. 10 is a schematic diagram illustrating a LEP having multi-spectra LED;

FIG. 11 is a schematic diagram illustrating a second embodiment of the LEP having multi-spectra LED;

FIG. 12 is a schematic diagram illustrating a LEP having multiple illumination sources; and

FIG. 13 is a schematic diagram illustrating a LEP having multiple fiber optic array from a single illumination source.

### DETAILED DESCRIPTION OF THE INVENTION

The invention is a low power, wearable, non-coherent illumination device (ID) using Light Emitting Diode(s) (LED). Also, the invention includes design improvements for a light emitting patch (LEP) that precisely places the LEP on a patient for 24 hours without hindering movement and without wearing cumbersome holding devices as well as handling fluids that may be near the illumination treatment area.

The initial design investigation of the ID was to determine where best to place the ID or the LED(s) of the ID in relation to the LEP and the LEP's light emitting surface (LES). First, the illumination (a single LED or an array of LEDs) at the bottom of the LEP's LES was explored such that the ID would also be near the treatment site of the LEP. This design, although attractive in simplicity did have two problems for long therapy times. One, it moved a source of potential heat close to the treatment site and two, it didn't provide the same capability for scaling the bandage in size without having to scale the size of the ID.

Based on the above design considerations (heat and scalability), the LEP design forced the ID location to be placed away from the LEP and from the treatment site to a place where the unit can comfortably sit. It was found that RSI can easily place the ID on the belt line of a patient. With the illumination further away, it required that RSI transport the light from the belt line to the treatment site, with the easiest method to use the fiber optics in the LEP as this transport mechanism. Again, to simplify the design, the easiest method to move light from the ID into the fibers was to bring the fibers into a circular bundle that can butt-couple to the ID LED illumination source. To properly butt-couple the fiberoptic circular bundle and the optic of the LED (used to shape the illumination from the LED die and to match the numerical aperture, acceptance angle, of the fiberoptics) a spring-switch (canted-coil assembly) described below is used.

The spring switch provided several functional features to the unit. It properly located the LEP input to the ID LED, it provided a locking mechanism that keeps the LEP in place during treatment but also allows (with 3-5 lbs. of force) the User to easily remove the LEP from the ID, it provides 360-degree of rotation of the LEP in the ID, and it also creates a switch that will only light to exit the LED when the LEP is locked in place. By making the LEP and ID become a switch, the potential risk of the high intensity LED light source exiting the ID when there is no LEP coupled to the ID is reduced.

The illuminator uses off-the-shelf (OTS) commercially-available LED light source modules. A laser can be used but the LED is sufficient for low-power output applications and the light does not need to be coherent, although it could be if needed.

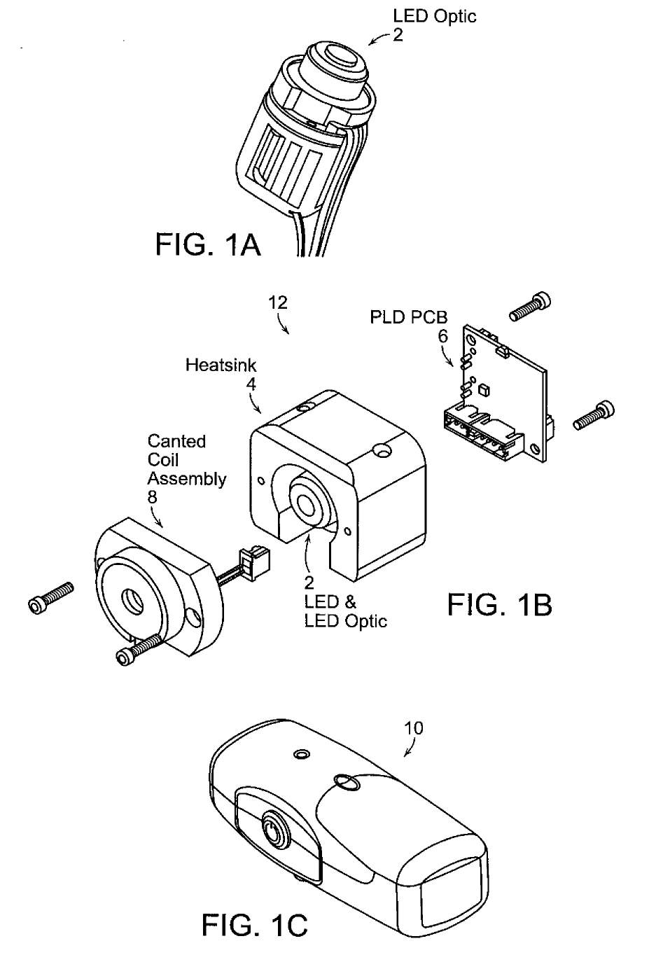

In the prior art, the illumination was pigtailed into a fiberoptic that was then expanded into a collimating optic which then was coupled into the fiberoptic patch array of the LEP. With the LED it was desired to remove the $250+ collimating optic. The invention provides a simple solution to shape the beam of light from the LEDs into a uniform field using an inexpensive plastic LED optic 2, as shown in FIGS. 1A-1B. Residing above the optic, the LEP would be butt-coupled to the output of the lens. FIG. 1B shows the LED optic 2 being mounted in a Heatsink 4 with RSI PCB 6 (upper right) attached to the back of the Heatsink and Spring/Switch (canted coiled assembly) 8 attached to the front of the Heatsink 4.

The ID unit 12 is designed using the LED optic 2 that was built on the square printed circuit board (PCB) 6. The LED optic 2 being placed on a round PCB 6 which is 0.81″ (20.5 mm) in diameter that allowed the ID to shrink in size and to enable the housing 10 to have a more flash-light shape and feel to the unit, as shown FIG. 1C.

Table 1 details the specifications on the LED module chosen for the ID system of FIG. 1B, which has a smaller footprint (mechanically and optically).

TABLE 1
LED specifications for the ID Rev 1 and Rev 2 electro-optic designs.

| | RSI ID Rev 1 | RSI ID Rev 2 |
|---|---|---|
| IOI LED Module | 2400B-100 | 2400B-250 Compact |
| # of Die | 1 and 4 | 4 |
| Numerical Aperture | 0.656 | 0.602 |
| Half Angle FOV | 41-deg | 37-deg |
| Output Diameter | 8.0 mm | 5.0 mm |
| (with Lens) | | |
| Power | 0.6 W (3 V, 0.2 A) | 0.6 W (3 V, 0.2 A) |
| Size | 1.5″ Long × | 0.81″ Diameter × 0.60″ Tall |
| | 1.0″ Wide × | |
| | 0.61″ Tall | |

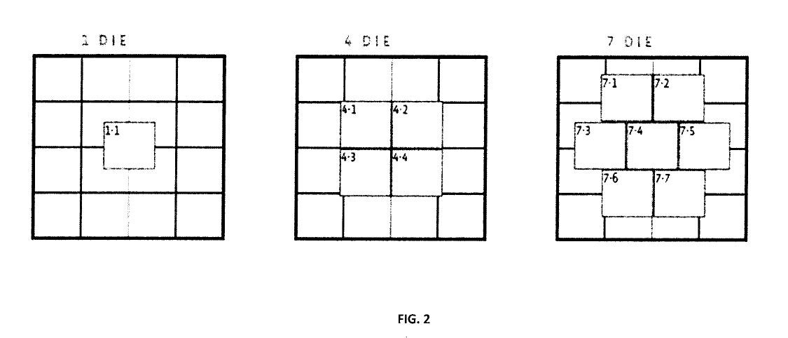

The ID unit 12 of FIG. 1B can be configured for use with 1-die, 4-die, or 7-die as shown in FIG. 2. Note in other embodiments of the invention there can be more dies used. By having more die, the spatial uniformity can be improved due to the Gaussian irradiance profile of each die (higher irradiance in the center with lower irradiance on the sides) and more light can be emitted from the LED. However, the tradeoff of having more die is more thermal heat to dissipate and an increase in cost so one must take this into consideration when designing an ID unit.

This locking mechanism 8 is a novel feature in the design of the ID unit 12 because it minimizes the risk of patient eye and thermal safety when the device has been turned ON by the User. The LED provides significantly more irradiance (˜1.4 mW/cm2) than the required 580 uW/cm2 that exits the 10 cm×10 cm output surface (LES) of the LEP due to light losses inherent in the system. This loss primarily comes from the butt-coupling of light from the LED to the LEP fiberoptics. Although the output of the LED without attachment to the LEP was deemed safe (through benchtop testing), it was imperative that patients do not: 1) look directly at the ID LED when the unit was turned ON and 2) try to use the device for other illumination needs beyond the phototherapy treatment. By placing a locking mechanism/switch into the unit, it locks the LEP into a well-defined standoff distance from the LED optic.

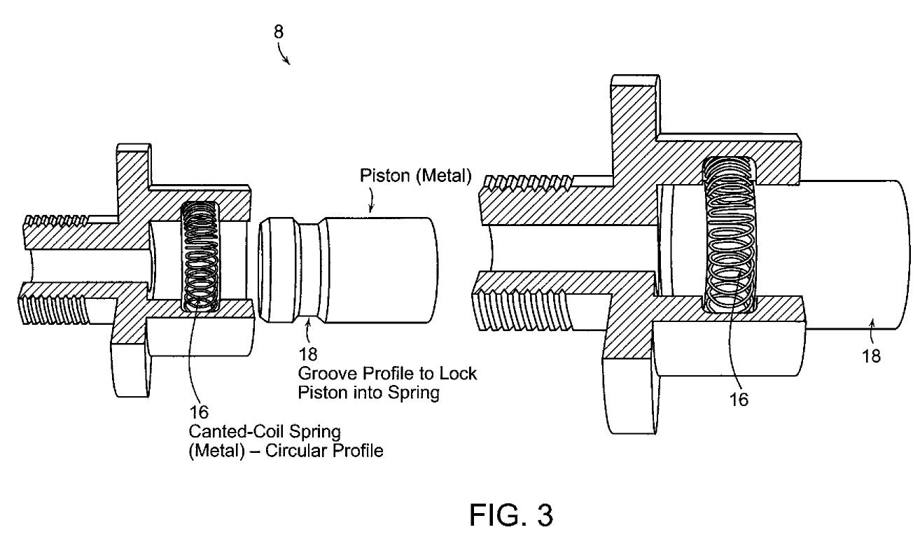

The locking mechanism or canted coil assembly 8 is composed of a canted-coil spring, a circular spring which is designed to produce a nearly constant force over the working spring deflection as opposed to traditional springs which have a spring force directly proportional to deflection. Canted coil springs can be used for latching, locking, and holding applications and can be customized based on diameter, connection (insertion and removal) force, and application use. An example of the canted coil spring 16 and its locking mechanism 8 is shown in FIG. 3. Notice that a piston 18 has a specific groove profile that once the piston is inserted into the spring, which is flexible to allow the piston to spread, the piston will be held in place. The insert and removal force required by the user can be tailored to the application. A canted coil spring, used in accordance with the invention would require an insertion and removal force of 5-lbs which is manageable for most patients.

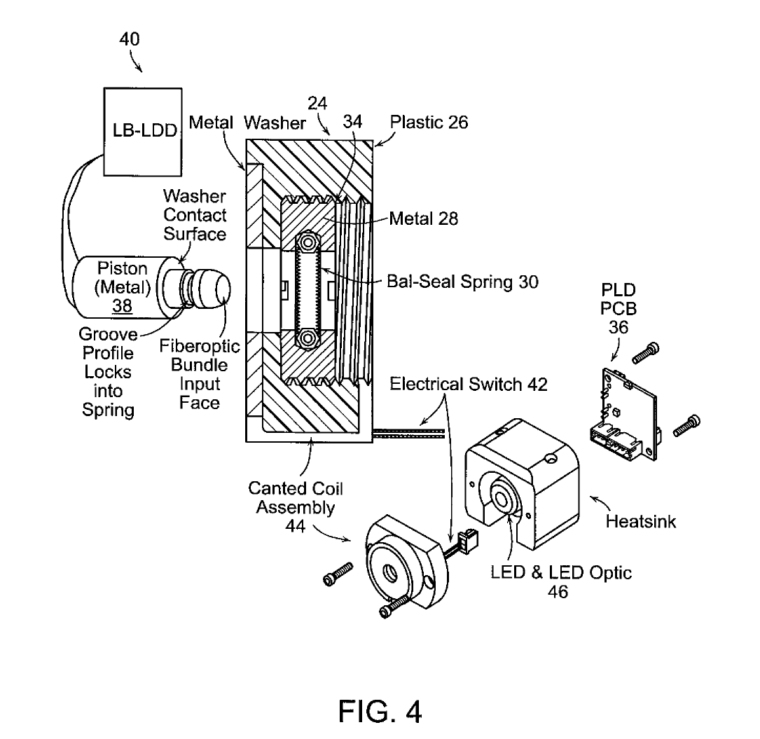

FIG. 4 shows the assembly in an ID unit 24 which has actually been produced and integrated into the fabricated ID. The ID unit 24 is composed of a medical grade plastic 26 that is electrically inert. There is a stainless steel metal holder 28 that holds the metal canted-coil spring 44. On top of the plastic holder is a stainless steel metal washer 32. There are electrical wires 34 connected to the metal washer 32 and metal spring holder 30 that run to the PCB 36 and shown in FIG. 4.

When the stainless steel piston 38 on the LEP 40 makes contact with the washer 32 and the metal spring holder 30, an electrical connection or switch 42 is made through the PCB 36 which can detect when this connection has been made. If the ID 24 is ON and the electrical connection is made, the LED 46 will be permitted to emit light. To make this electrical connection when the ID is ON, the metal contact surface of the LEP piston 38 has to maintain contact with the metal washer 32. A key benefit of this locking design is that it eliminates the user from having to directly control any shuttering, it avoids cumbersome switches, and it allows the LEP 40 to swivel an entire 360-degrees around the ID 24 because the LEP piston 38 can rotate freely within the spring 30 while maintaining a locking force and surface contact with the washer 32.

The ID units used in accordance with the invention can use D-Cell sized battery with significantly higher voltage (3.6V) and current (13.0 Ah) ratings than a standard commercial grade D-Cell battery and which can provide the required energy needed for 24 hours of CLIPT treatment. The battery is composed of Lithium Thionyl Chloride (Li—SOCl₂) and RSI found several manufacturers with these batteries that had been tested to UL electrical and safety requirements. RSI's Rev 2 ID has since incorporated the Li—SOCl₂ battery design and has undergone initial bench testing.

To control the power input and output of the battery, the LED and LED PCB, and the User functionality of the ID, the PCB used firmware and hardware controls to operate the device. The PCB, with double-sided populated electronics was shrunk to 1″×1″ and was attached to the back of a heatsink holding the IOI LED.

A main feature of the PCB is that all of the main components of the ID comprised of the battery connection, the power switch, the LED, and the spring-switch all connect to the PCB via Molex connections. Each part can be built separately and then attached at the end to the PCB before closing the entire assembly with the ID housing. The design of the PCB also allows the user to adjust the power output for future clinical studies that can explore higher or lower energy delivery by adjusting a potentiometer. Additionally, the design of the unit accounts for several time settings allowing us to turn Off the device at varying time settings including 15 minutes, 30 minutes, 45 minutes, 1 hour, 2 hours, 4 hours, 6 hours, 8 hours, 12 hours, and 24 hours.

The mechanical housing design was carefully designed to account for five interaction surfaces. 1) The housing made accommodations for the push-button power On/Off such that it can rest on one of the inner housing molds so that the unit can be assembled much easier. 2) The housing placed a small fiberoptic port over the LED Status light on the PCB to allow the User to see the LED Status indication light with clarity. 3) The battery door was designed several times using SLA molds and mock ups to get a comfortable but stable latching for User interaction when inserting and removing the battery. 4) The entrance port for the LEP was improved to allow the LEP to swivel around the ID to avoid any cable tangling and the entrance port was deepened to mitigate any risk from electrical contact with any switches, including the spring-switch. 5) The back of the ID was given some additional feature contours where a belt clip can be added to the unit.

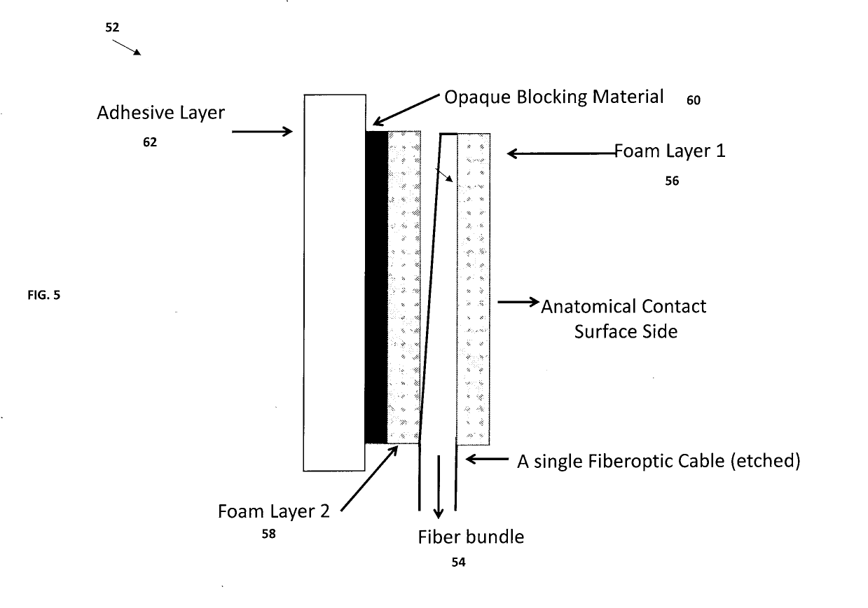

The basic design of a LEP 52 can be seen in FIG. 5. The unit 52 is composed of several key elements starting with a fiber optic cable (FOC) array, composed of one or more fiber optic cables that are typically less than 1 mm in diameter. These optical fibers 54 can be plastic or glass.

The FOCs 54 in the array are typically placed in a one-dimensional (1D) arrangement where each FOC 54 in the array is parallel to one another. The placement of one FOC to another can be abutting, equally spaced, or non-linearly spaced. Each FOC is cut typically to a length of 24-48″ long to reach multiple locations on the body once the LEP is finally assembled.

The FOC 54 is commonly laid onto medical grade adhesive backed foam layer 56, a polyethylene foam single coated medical tape. Each FOC 54 will make contact and stick to the adhesive side of the foam layer 56. The foam layer 56 is usually cut as a square ranging in size, but a typical size is 100 mm×100 mm.

The foam/FOC layer is then attached to a fixed mechanical fixture inside a laser cutter. The mechanical fixture may be a vacuum chuck. A laser program that will deliver the optimal 1-D, 2-D, or 3-D fiber etch will be entered and run.

Following the etching of the fibers, the foam/FOC layer is removed from the laser cutter and will undergo further assembly. A second foam layer 58, which can be similar to the first foam layer 56 is placed on top of the open and exposed area on the FOC 54. Typically, on top of this second layer 58 is an opaque covering 60, like a black polyester sheet to block any light from exiting the LEP towards the user and other external observers. Note any one of the layers can have a diffusing patter or a diffuser on it to help aid in the light structure output that can make the light more uniform or to make custom patterns for a given application/—including wound care.

Typically a final layer 62 is added composed of an adhesive backed (acrylate adhesive developed for medical/surgical use) medical nonwoven tape. The adhesion of the material is 30 oz./inch which will allow it to stick a patient's skin or to other bandage and cast linings for long periods of time. The adhesive layer 62 can be cut in the middle to allow the foam/FOC/foam layer to be placed in the center and then covered with the adhesive layer's 62 liner, a poly-coated Kraft paper with silicone release on one side.

There are typically several epoxies used to lock down or tighten surfaces. Epoxy is typically used to adhere the bottom of the foam/FOC/foam layers with the Fiber Transition part as well as the silicone rubber sheathing with the Fiber Transition part and the piston. Fibers in the stainless steel piston are locked in place using EPOTEK 301 Epoxy and a potting station. After all the FOC 54 are rigid and in place, a diamond cutting device is used to sharply cut the fibers to have a common optical interface surface with few surface aberrations or minor surface deformations.

An important feature of the LEP is that it has bend radii in both the vertical and horizontal dimension that is less than 1.0 cm allowing for extreme flexibility on array of anatomical features while consistently providing constant illumination along these tight bends.

Another important feature of the LEP is the ability of the system to stick or adhere to skin or other objects by attaching fiberoptic cables and any surrounding layers that hold the fiberoptic channels in place to medical grade adhesive materials, epoxies, glues, or gel forming substrates.

Another important feature of the LEP is the absorption properties of the many layers holding the fiberoptic cables. These layers can absorb or wick away blood, fluids, and other substances that may result from a wound. These layers also allow the fiberoptics to remain clean and deliver light during wound care treatment. Additional methods to keep the fiberoptic cables clean may include the release of alcohols, solvents, or powders to reduce fluid buildup or drying blood, fluids, or substances on the fiberoptics. The spacing of the FOCs also helps pass blood, fluids, and substances past the fiberoptics and into the adjacent layers, particularly the foam layers that have absorption properties.

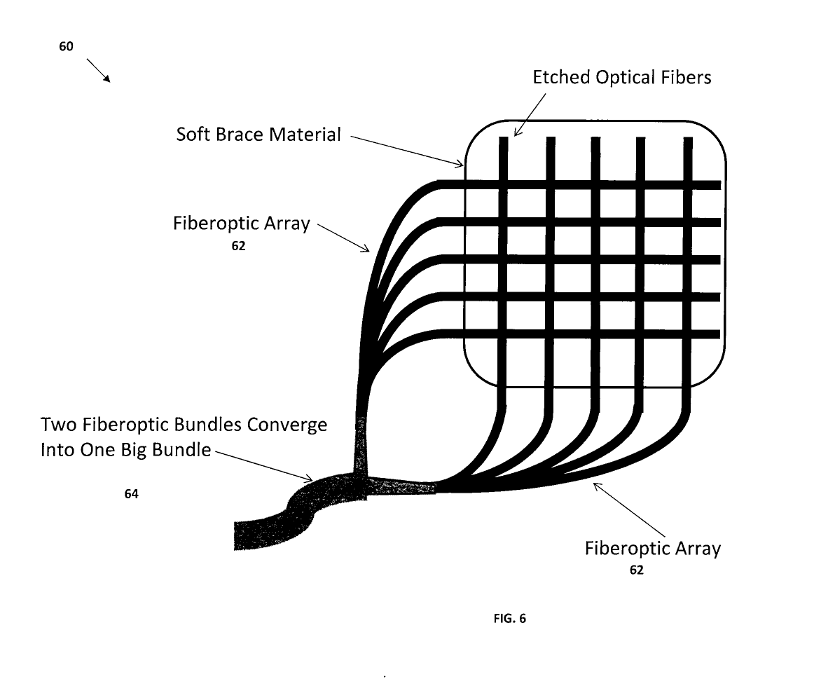

There are many different permeations of the LEP. FIG. 6 shows a LEP 60 having a multitude of fiber optic arrays 62 can be placed along a soft brace material such as a bandage, cast, or lining to create more device flexibility or to output varying light patterns. These fiber optic arrays 62 can be brought together into a common ID via a bundle 64 that can deliver multi-spectral and UV illumination. Each array 62 may have light input from the ID that is of only one spectrum which will exit the array on the LES of the LEP. Each array may receive light input from the ID that is of one or more spectrum which will exit the array on the LES of the LEP. The ID can contain one or more LEDs to deliver light to the fiber optic arrays and the LEP. Note each LED can contain multiple die which can be composed of and emit multiple wavelengths

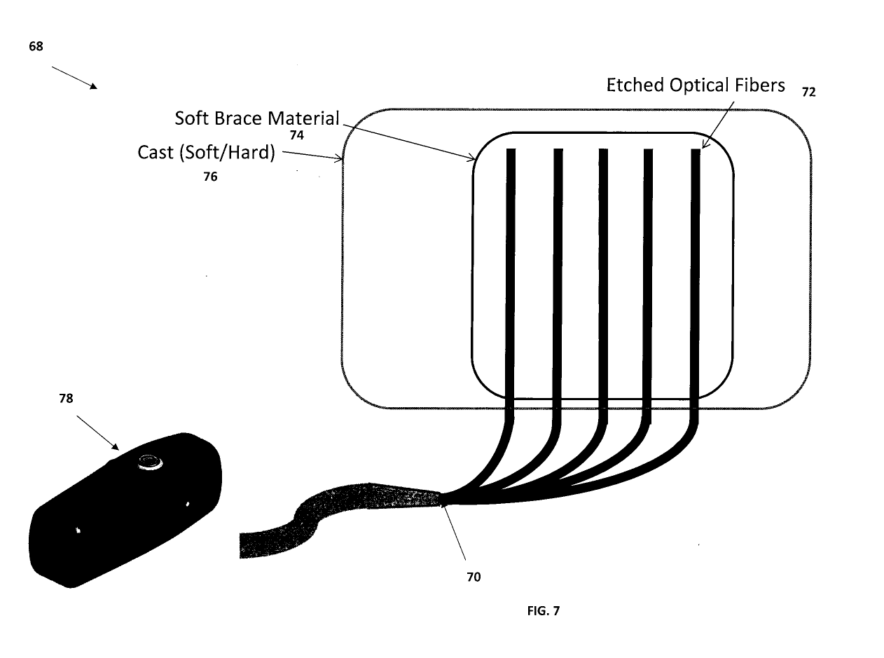

FIG. 7 shows another embodiment of the LEP 68 where there is only one fiber optic array 70 but it can be composed of one or more arrays with each array consisting of one or more fiber optic cables 72. The fiber optic array 70 is attached to a soft brace 74 material which is attached or integrated into a hard or soft cast 76. The fiber optic array is coupled to an ID unit 78.

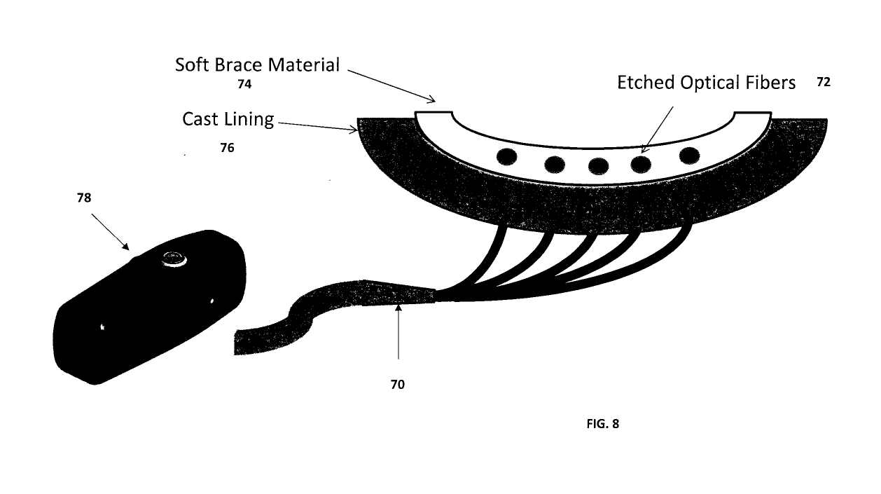

FIG. 8 shows the top-view profile of the layout of the fibers 72 and the materials shown in FIG. 7.

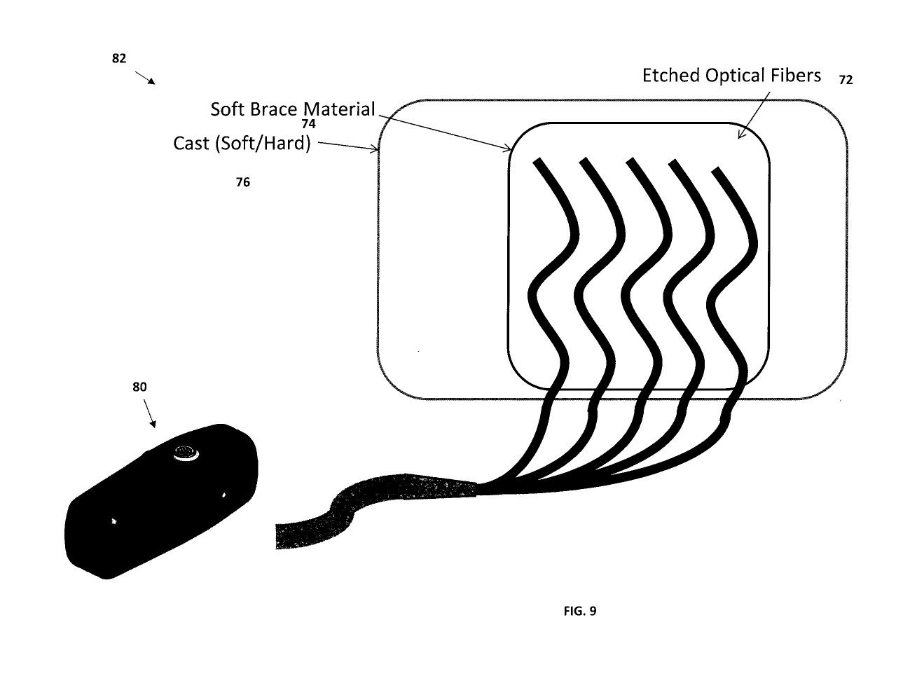

FIG. 9 shows an LEP 82 having the same LEP 68 of FIG. 7 except each fiber optic cable 72 can have varying bending patterns to make the surface of the LEP and the LES to be more flexible. Each fiber optic cable is bending several times back and forth. The bending pattern can be applied to the fiber optic by mechanical stressing, radiation stressing, or thermal stressing. The bending pattern is applied by shape molds that will form the fiber to a given pattern. Each fiber optic cable in an array can be composed of one or more bending or shape patterns.

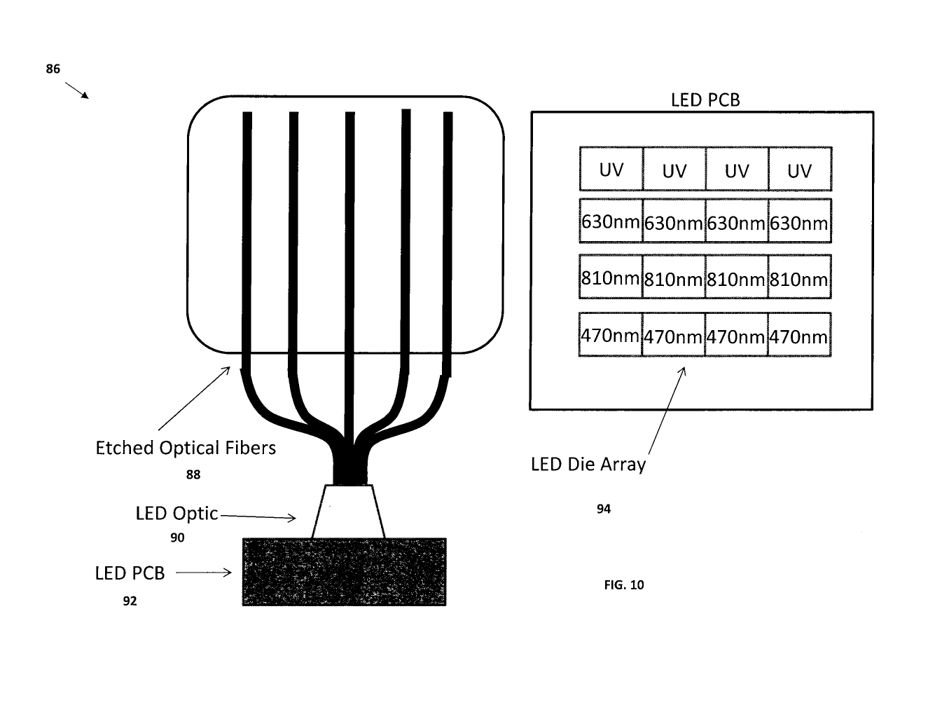

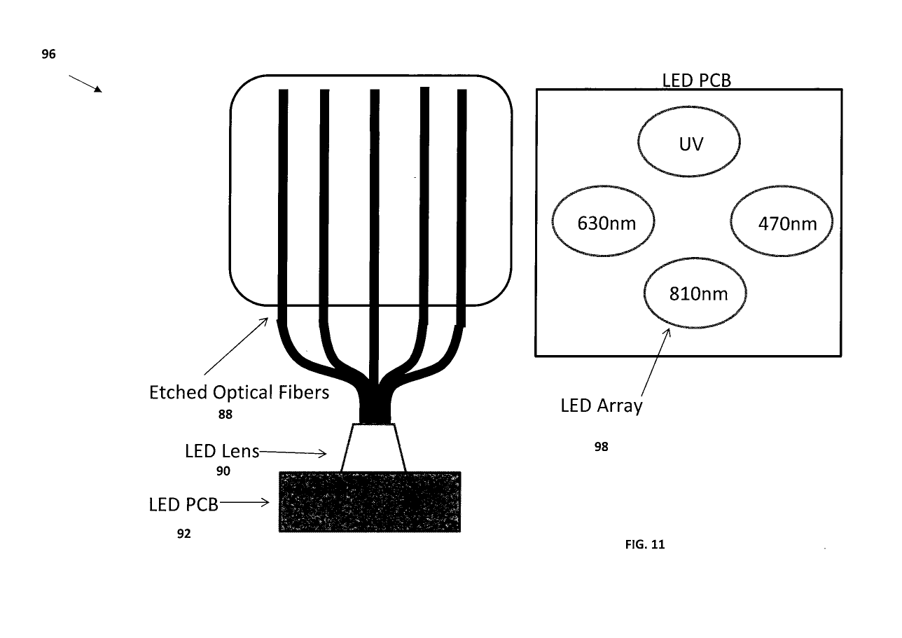

FIGS. 10 and 11 show LEP arrangements single fiber optic array 88, 96 attached to a LED module where the fiber optic array 88 is butt-coupled to the LED Optic 90. The LED is composed of an array of LED die arrangements 94 that reside on a LED PCB 92. FIG. 10 shows a 16-die configuration 92 where one row (of four die) is composed of UV, a second row of LED die 92 are composed of multi-spectral illumination in the blue wavelengths at approximately 470 nm. Another row of LED die 92 is composed of multi-spectral illumination in the red wavelengths at approximately 630 nm. Another row of LED die is composed of multi-spectral illumination in the near-infrared wavelengths at approximately 810 nm. In FIG. 10, the wavelengths can vary as can the die configuration and the number of die.

FIG. 11 shows a LEP arrangement 96 having has a similar layout as FIG. 10 except on the LED PCB 92 is four separate LED sources 98 for the UV, 470 nm, 630 nm, and 810 nm. Each separate LED source 98 can be composed of an array of LED die of varying illumination intensity, wavelength, and delivery rate (continuous or periodic/pulsing). The device in FIG. 11 can be composed of varying wavelengths and each array of die at each LED source 98 can be configured in different patterns.

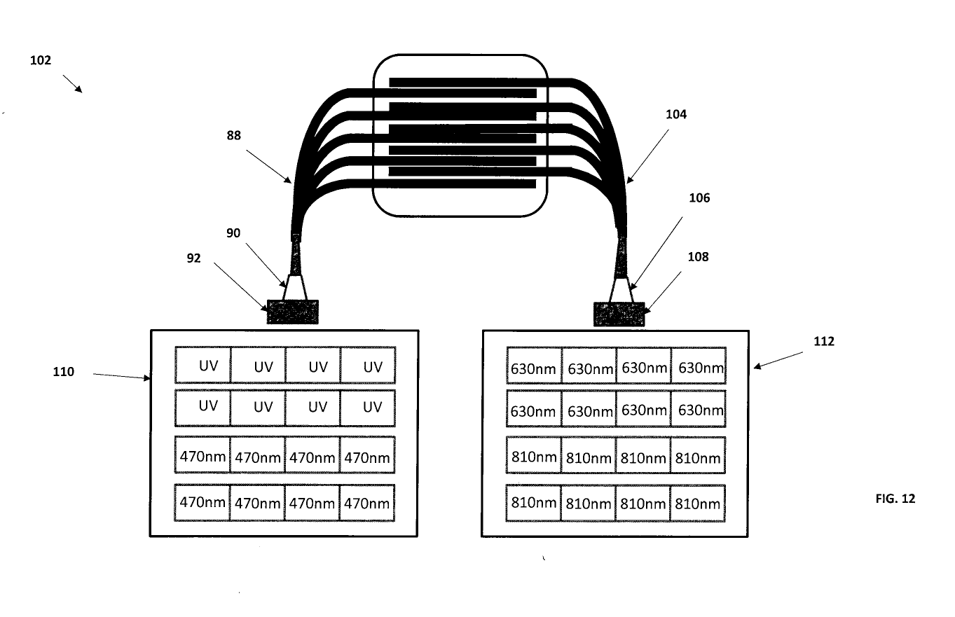

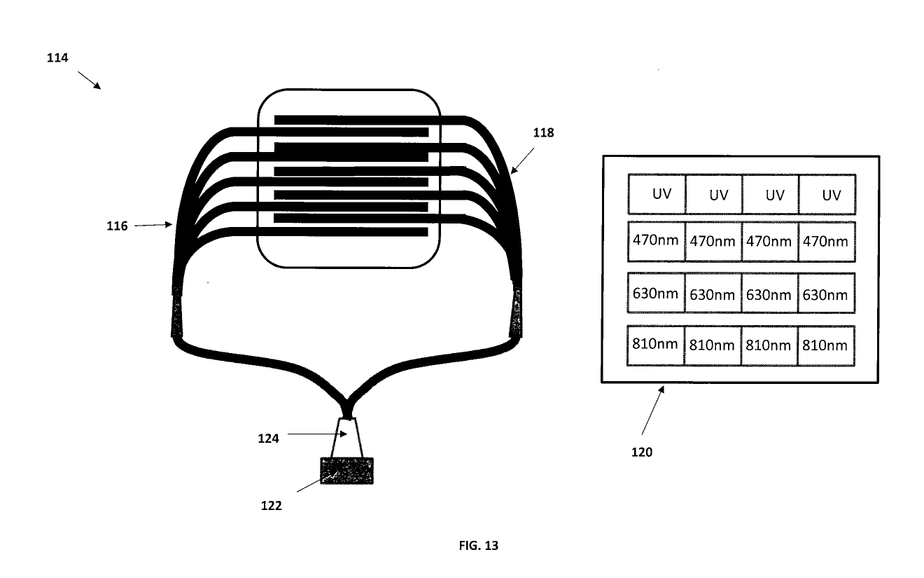

FIG. 12 shows a LEP 102 having a similar layout to the LEP 86, 96 of FIGS. 10 and 11 except one or fiber optic arrays 88, 104 can be directed to one or more LED sources 110, 112 on one or more LED PCBs 92, 108 on one or more IDs. Splitting the fiber optic arrays 88, 104 allows for more LED die to be concentrated on a given array at a given spectrum or spectrums. As an alternative, FIG. 13 shows a LEP 114 having a multitude of fiber optic arrays 116, 118 coupled to a single LED source 120 and LED PCB 122. The coupling between the LEP 114 and the ID's LED optic 124 can be through butt-coupling or by other opto-mechanical methods that will optimize the coupling of the LED illumination into each fiber optic cable.

Note all the IDs described herein allows for the control of an illumination pattern by controlling the duration of the illumination, duration that wavelength is on/off, duration that a given wavelength is on/off per treatment, duration that a given wavelength is on/off per day, variation of irradiance (or Intensity) of each wavelength of light when a given wavelength is On, coordination of wavelengths when they're On, pulsing or continuous light output of any given wavelength, and variation in illumination pattern on the wound site from each wavelength.

The invention can utilize alternative embodiments or enhancements. For example, the light diffusion technology and fabrication process can be used to etch fibers that are embedded into bandages with or without adhesive. The fibers can be pre-etched and then adhered to either the adhesive or non-adhesive side of an adhesive bandage or applied directly to a non-adhesive bandage. The pre-etched fiber can also be embedded into the bandage. Alternatively, the fiber can be placed on any surface of a bandage or embedded in the bandage and then the bandage and fiber can be cut by the laser etching process. The laser etch cut may allow for mechanical features of the bandage while also creating the light diffusion pattern on the fibers.

The fibers can be placed on any surface of the proposed bandage or embedded but may be flexed in various geometric bending positions or be wrapped in circular loops to provide more flexibility to the bandage. These complex shapes can help provide various mechanical and human factor conditions that may not be met with a straight fiber.

The bandages with pre-etched or post-etched fiber optics cables can of many different aerial sizes but would ideally be 1 cm2, 5 cm2, 10 cm2, and 20 cm2 in size. The bandages with etched fibers can receive light from a fiber from an LED or laser light source. The fibers in the bandage can have a common input at one end of the bandage allowing for the coupling of the light through additional fiber optics or various other optical systems. The light bandage could be used in similar applications and medical indications used throughout this application.

The light bandage can receive light from a LED cartridge system. An array of LED modules can be coupled to a given bandage consecutively or simultaneously. The LED modules are worn around the shoulder like a harness or on the belt-line.

The layers described herein can be composed of a multitude of other layers or materials. The stack configuration of the layers can vary depending on the application. The number of layers can vary. One or more layers may provide additional features to the bandage, dressing, liner, cast, or fabric. For example, one layer may consist of a drug eluting surface that can release drug continuously or on a periodic basis. Drugs that might elute from one or more surfaces include nanoparticles, photosensitizers, antimicrobial drugs, oncology drugs, and other ointments. One or more layers may include antimicrobial coatings. One or more layers of the LEP may include microneedles that can puncture the skin using micron sized needles. These needles can deliver light, drugs such as photo sensitizers, or a combination of the light and drugs. The drugs can elute from the needles continuously or periodically.

The illumination device attached to the LEP can emit UV (260-400 nm), Visible to Near-Infrared (400-1000 nm), Near-Infrared (900-1700 nm) and Short-Wave Infrared (900-2500 nm) with the potential for Mid-Wave and Long-Wave Infrared illumination (2500 nm to 17,000 nm). The LEP or LEP can transport and delivery illumination over this spectral range of 260 nm to 17,000 nm. The illumination from the ID and the LEP LES can range from 0.001 mW/cm2 to 10,000 mW/cm2.

There are a multitude of other light delivery mechanisms that could be used to create the LES of the LEP. This can include new transparent polymer materials, silk, or new synthetic carbon grapheme or nanotube particles that can emit light.

In conjunction with the LEP and ID, there can be the addition of a monitoring sensor to monitor light input/output from the LEP fibers but also from the patient's anatomy or skin. The sensors can be used to monitor and control light dosimetry based on thermal criteria, bacteria load criteria, fluid levels, or other human-environmental conditions that may affect the healing process.

There are an array of other potential application for the LEP or LES. For example, the LEP can be combined with negative pressure wound therapy dressings to create a more effective therapy. In this use-case scenario, the vacuum dressing would be laced with etched optical fibers. The unit would be controlled with a single device that includes a negative pressure pump and an illumination device merged into one device.

The LEP and the etched fiber optic cables can also be laced into a traditional gauze for treating less severe chronic wound management needs. The LEP can use an optically transparent non-adhesive dressing such that light can be delivered external to the wound dressing, cast, lining and fabric to promote light mediated wound therapy. In this configuration, the light can be delivered by etched fiber optic cables or it could be delivered by an illumination source other than a fiber optic cable, such as an LED.

The transparent dressing would have several benefits such as helping reduce thermal issues, reducing the device or fibers from being in direct contact with wounds, providing improved sterility, and reducing biocompatibility issues. The LEP layers, including the transparent dressing have the potential for providing liquid growth factors in conjunction with the bandage to promote growth and healing faster and more effectively without the LEP or its material properties. The LEP can be used in brace lining for backs, knees, and legs.

The invention can be applied to traditional wound dressing to assist acute wound treatment. Minimally invasive surgery has replaced many open surgical procedures. Infections in the skin portals that communicate between the outside environment and internal body cavities, while rare can be devastating. In particular, the percutaneous power supply for left-ventricular assist devices (LVAD) is a frequent portal for infection that can be life-threatening. Patients often develop multiple drug-resistant infections due to the frequent and chronic use of antibiotics to prevent and treat these percutaneous line-infections. The invention can be configured to work with vacuum-dressings or conventional pressure/compression dressings.

Moreover, the invention can applied for treating diabetic ankle and foot ulcers, pressure/decubitus ulcers, soft casts—bone healing, as well as other similar remedies.

Although the present invention has been shown and described with respect to several preferred embodiments thereof, various changes, omissions and additions to the form and detail thereof, may be made therein, without departing from the spirit and scope of the invention.

What is claimed is:

1. A therapeutic light delivery system comprising:
one or more fiber optic arrays that channels illumination from an illumination device;
an optics module coupled to the one or more fiber optic arrays that provides the illumination to the one or more fiber optic arrays; and
a control module coupled to the optics module that control the illumination to the one or more fibers, the illumination comprises a plurality of therapeutic illumination patterns and wavelengths.

2. The therapeutic light delivery system of claim 1, wherein the optic module comprises a LED optic and heatsink.

3. The therapeutic light delivery system of claim 1, wherein the control module comprises a control board.

4. The therapeutic light delivery system of claim 3, wherein the control module comprises a plurality of dies to control illumination for a LED.

5. The therapeutic light delivery system of claim 1, wherein each of the one or more fiber optic arrays comprise etched fibers.

6. The therapeutic light delivery system of claim 5, wherein the one or more fiber optic arrays comprise a single fiber optic array positioned on a soft brace material.

7. The therapeutic light delivery system of claim 1, wherein each of the one or more fiber optic arrays comprise bent etched fibers.

8. The therapeutic light delivery system of claim 1, wherein the one or more fiber optic arrays comprise a plurality of fiber optic arrays.

9. The therapeutic light delivery system of claim 7, wherein the control module is coupled to a plurality of illumination sources.

10. The therapeutic light delivery system of claim 9, wherein the control module is coupled to a plurality of dies for illuminating LEDs.

11. The therapeutic light delivery system of claim 8, wherein the control module comprises a plurality of control boards used to control illumination.

12. A method for delivering light for wound care comprising:
providing one or more fiber optic arrays that channels illumination from an illumination device;
coupling an optics module to the one or more fiber optic arrays that provides the illumination to the one or more fiber optic arrays; and
controlling the illumination to the one or more fibers using a control module coupled to the optics module, the illumination comprises a plurality of therapeutic illumination patterns and wavelengths.

13. The method of claim 12, wherein the optic module comprises a LED optic and heatsink.

14. The method of claim 12, wherein the control module comprises a control board.

15. The method of claim 14, wherein the control module comprises a plurality of dies to control illumination for a LED.

16. The method of claim 12, wherein each of the one or more fiber optic arrays comprise etched fibers.

17. The method of claim 16, wherein the one or more fiber optic arrays comprise a single fiber optic array positioned on a soft brace material.

18. The method of claim 12, wherein each of the one or more fiber optic arrays comprise bent etched fibers.

19. The method of claim 12, wherein the one or more fiber optic arrays comprise a plurality of fiber optic arrays.

20. The method of claim 19, wherein the control module is coupled to a plurality of illumination sources.

21. The method of claim 20, wherein the control module is coupled to a plurality of dies for illuminating LEDs.

22. The method of claim 19, wherein the control module comprises a plurality of control boards used to control illumination.
